(19) United States
(12) Patent Application Publication
(10) Pub. No.: US 2012/0259200 A1
(43) Pub. Date: Oct. 11, 2012

(54) Method for Monitoring Early Treatment Response

(75) Inventor: Joseph F. Norfray, Glenview, IL (US)

(73) Assignee: Receptomon, LLC, Glenview, IL (US)

(21) Appl. No.: 13/439,216

(22) Filed: Apr. 4, 2012

Related U.S. Application Data
(60) Provisional application No. 61/471,965, filed on Apr. 5, 2011.

(51) Int. Cl. A61B 5/055

(52) U.S. Cl. 600/410

(57) ABSTRACT

Disclosed is a method for monitoring early treatment response of a cancer treatment comprising measuring by magnetic resonance spectroscopy (MRS), for example, ¹H MRS, the amount of saturated and unsaturated fatty acids present in the intracellular membranes of the cancerous tissue before and after treatment, whereby an increase in the amount of saturated and unsaturated fatty acids after treatment is indicative of a positive response from interruption of protein translation. The increase in the amount of saturated and unsaturated fatty acids represents the degradation in the internal cell membrane as a result of down regulation of the organelles and their secretory granules and their transport vesicles. Disclosed also is a method for determining effectiveness of a cancer treatment to induce apoptosis from interruption of protein translation in an animal having a cancerous tissue by MRS.

### CROSS-REFERENCE TO RELATED APPLICATIONS

This application claims the benefit of U.S. Provisional Patent Application No. 61/471,965 filed on Apr. 5, 2011, the disclosure of which is incorporated herein in its entirety by reference.

### BACKGROUND OF THE INVENTION

Some of the common approaches to cancer treatment include surgery, radiation therapy, and chemotherapy. Radiation therapy and chemotherapy are effective if they are capable of killing the tumor cells, for example, when they act as cytotoxic agents. Typically, the response to radiation therapy or chemotherapy is monitored by magnetic resonance imaging (MRI) of the tumor, wherein a decrease in tumor size is indicative of positive response to treatment.

While MRI is a tool for monitoring treatment response, the disclosures in the art show that in many cases, a detectable change in tumor size is observed only after a significantly long period of time, for example, after treatment for a period of about 3 months or more. Such long periods of time could be harmful to the patient, especially if the treatment has not been effective or only partially effective, such as, for example, treatments involving the use of peptide inhibitors; during this long period of time, tumor cells could multiply or metastasize, and lead to worsening of the patient's condition.

Magnetic Resonance Spectroscopy (MRS) has been proposed as a tool for obtaining information on cellular metabolism; see, for example, Norfray, J. et al., Ch. 110 in Pediatric Neurosurgery, 4^{th} ed., McLone, D. G., et al. (Eds), W. B. Saunders Co. (2001). MRS also has been proposed for diagnosing the treatment response of tumors with cytotoxic agents; see, for example, Fulham, M. J., et al., Radiology, 185, 675-686 (1992), which discloses that brain tumor metabolism was studied with ¹H MRS before and after treatment with radiation therapy. MRS permits non-invasive examination of metabolic characteristics of human cancers in a clinical environment. Accessible nuclei include ³¹P, ¹³C, ¹H, and ²³Na. ³¹P MRS contains information about energy status (phosphocreatine, inorganic phosphate, and nucleoside triphosphates), phospholipids metabolites (phosphomonoesters and phosphodiesters), intracellular pH (pH NMR), and free cellular magnesium concentration (Mg²⁺ f). Water-suppressed ¹H MRS shows total choline, total creatine, lipids, glutamate, inositols, lactate, and the like. Negendank, W., NMR in Biomedicine, 5, 303-324 (1992).

Further, U.S. Pat. No. 6,681,132 (Katz et al.) discloses a method for determining the effectiveness of chemotherapy comprising administering a dose of a cytotoxic antineoplastic agent to a subject prior to surgical removal of a cancerous tumor, acquiring magnetic resonance data from the subject, and determining whether the treatment has affected the population of a nuclei, particularly ²³Na. Negendank, W., supra, provides a review of various studies of human tumors by MRS.

See also Ross, B. et al., The Lancet, 8378, 641-646 (1984) discusses monitoring response to cytotoxic chemotherapy of intact human tumors by ³¹P MRS; Griffiths, J. R. et al., The Lancet, 8339, 1435-36, Jun. 25, 1983 discloses the use of ³¹P MRS to follow the progress of a human tumor during chemotherapy with doxorubicin; Ross, B. et al., Arch. Surg., 122, 1464-69 (1987) discloses the monitoring of chemotherapeutic treatment response of osteosarcoma and other neoplasms of the bone by ³¹P MRS; and Norfray, J. F. et al., J. Computer Assisted Tomography, 23(6), 994-1003 (1999) discloses a 1 MRS study of the neurofibromatosis type 1 intracranial lesions.

The goal of all successful cancer therapies is to cause apoptosis, a genetically controlled programmed cell death. (1) Schmitt, C., A. et al., J Pathol 187, 127-187 (1999); Ferreira, C. G. et al., J Clin Res 8, 2024-2034 (2002); Hotchkiss, R. S. et al., N Engl J Med 361, 1570-1583 (2009). Cytostatic, cytotoxic (chemotherapy and radiation), as well as, antiangiogenesis inhibitors therapies cause apoptosis. During apoptosis, DNA, organelles (the intracellular membranes) and cytoskeleton are degraded by specific enzymatic catalytic proteins, while maintaining the plasma membrane, thereby, preventing release of the inflammatory degradation products. Apoptosis has two functional phases: the early commitment phase where proteases and lipases are generated, and the late execution phase where the targeted plasma membranes containing degraded byproducts are phagocytized.

U.S. Pat. Nos. 7,289,840 and 7,622,102 and U.S. patent application Ser. Nos. 11/622,321 and 11/397,877 disclose a method for monitoring early treatment response of a cancer treatment comprising measuring by magnetic resonance spectroscopy (MRS), for example, proton MRS, the amount of Choline present in the cancerous tissue before and after treatment; the treatment comprises administration of a cytostatic, antiangiogenic, and/or cytotoxic therapies, whereby a decrease in the amount of Choline after treatment is indicative of a positive response. The decrease in the amount of Choline represents the decrease in the internal cell membranes as a result of down regulation of the organelles and their secretory granules and their transport vesicles. Disclosed also is a method for determining effectiveness of cytostatic, antiangiogenic and/or cytotoxic therapies in the treatment of cancer. Further disclosed is a method for monitoring protein translation in an animal having cancerous tissue by MRS.

Attempts have been made to study the source of ¹H MRS peaks of saturated/unsaturated fatty acids at 1.3, 2.8 and 5.4 ppm following treatment. These peaks have been ascribed to metabolites of unknown degraded lipids within intracellular lipid droplets and intracellular vesicles; Lindskog, M., et al., J Natl Cancer Inst 96, 1457-1466 (2004), Hakumaki, J. M., et al., Natl Med, 5, 1323-1327 (1999), Delikatny, E. J., et al., Cancer Res, 62,1394-1400 (2002), and Kauppinen, R. A., et al., NMR Biomedicine, 15, 6-17 (2002).

The foregoing shows a need to identify the source of MRS peaks of saturated/unsaturated fatty acids and is the basis of a reliable method for monitoring early treatment response.

### BRIEF SUMMARY OF THE INVENTION

The invention provides a method for monitoring early treatment response of a cancer treatment comprising measuring by MRS, the amount of saturated/unsaturated fatty acids present in the intracellular membranes of the cancerous tissue before and after treatment, determining whether the amount of saturated and/or unsaturated fatty acids have increased, decreased, or remained the same, and correlating an increase in the amount of saturated and unsaturated fatty acids after treatment to a positive response resulting from interruption of protein translation due to the treatment.

In accordance with the invention, diagnosed cancers can be monitored by following cell membrane metabolites of saturated/unsaturated fatty acid peaks on MR spectroscopy. The saturated/unsaturated fatty acid peaks represent bioassays of the visible mobile saturated/unsaturated fatty acids forming the plasma and organelle cell membranes. An increase in the saturated/unsaturated fatty acids identifies a positive treatment response.

The present invention provides several advantages, for example, the amount of saturated/unsaturated fatty acids change prior to classical imaging findings, and the MRS peaks corresponding to saturated/unsaturated fatty acids change with a treatment inducing apoptosis. The present invention offers the combined advantages of MRI and MRS and provides a method to monitor early treatment response. The present invention also provides a method for monitoring cancer treatment. In addition, the present invention provides for a method for monitoring interruption of protein translation in an animal having a cancerous tissue comprising administering a cancer therapy to the animal and measuring, by Magnetic Resonance Spectroscopy, the amount of saturated/unsaturated fatty acids present in the cancerous tissue before and after administering the cancer therapy.

### DETAILED DESCRIPTION OF THE INVENTION

This patent application is predicated on the MRS quantification of metabolites of intracellular membranes, the organelles. Intracellular membranes (organelles) are composed of both Choline polar heads, and saturated/unsaturated fatty acid tails, Ch 5 in Molecular Cell Biology, 5^{th} ed., Lodish, H., et al. (Eds), W. H. Freeman Co (2004). The present invention monitors components of organelles, namely, the saturated/unsaturated fatty acid tails to determine the effectiveness of a cancer therapy resulting from the interruption of protein translation. Norfray's U.S. Pat. Nos. 7,289,840 B2; 7,622,102 B2; and 7,771,706 B2; and U.S. patent application Ser. Nos. 11,576,198 and 11,397,877 have described a “Method For Monitoring Early Treatment Response: utilizing the Choline polar heads of intracellular membranes. This patent application describes a distinct “Method For Monitoring Early Treatment Response” utilizing the fatty acid tails of intracellular membranes. The Methods for Choline polar heads and fatty acid tails are distinct because the Choline and fatty acid peaks, trafficking and quantification are different.

The present invention is predicated on monitoring changes in the amount of one or more metabolites occurring in fatty acid tails of an internal cell membrane, for example, changes induced by the down regulation of one or more of the intracellular organelles and their secretory granules and transport vesicles. The internal cell membranes, which constitute nearly 90% of the total cell membranes, form the membranes of the nucleus, the mitochondria, the lysosomes, the peroxisomes, the endoplasmic reticulums, the Golgi apparatus, the secretory granules, and the transport vesicles. Successful cancer therapies down-regulate the intracellular organelles and their secretory granules and transport vesicles.

Accordingly, the invention provides a method for monitoring early treatment response of a cancer treatment comprising measuring by MRS, the amount of saturated/unsaturated fatty acids present in the intracellular membranes of the cancerous tissue before and after treatment, determining whether the amount of saturated and/or unsaturated fatty acids have increased, decreased, or remained the same, and correlating an increase in the amount of saturated and unsaturated fatty acids after treatment to a positive response resulting from interruption of protein translation due to the treatment.

The present invention provides a method for monitoring early treatment response of a cancer treatment comprising measuring, by Magnetic Resonance Spectroscopy (MRS), the amount of saturated/unsaturated fatty acids present in the cancerous tissue before and after treatment, whereby an increase in the amount of saturated/unsaturated fatty acids after treatment is indicative of a positive response.

MRS can be based on the resonance of any suitable nuclei; for example, nuclei selected from the group consisting of ¹H, ¹³C, and any combination thereof; preferably, ¹H.

An increase in the amount of saturated/unsaturated fatty acids occurs very early in the commitment phase of apoptosis from blockage of ribosomal assembly, and thus, protein synthesis, Clemens, M. J. et al. Cell Death Differ, 7, 603-615 (2000). Only the mobile fatty acids are visualized. Since up to 90% of the cell membranes can be down regulated in a tumor, MRS provides a sensitive method to monitor early treatment response.

In accordance with an embodiment of the invention, the term saturated fatty acids is used to denote the ¹H chemical shift of the CH₂ group at 1.3 ppm, for example, as in —(CH₂)ₙ—, where n represents the number of carbon atoms in the fatty acid, e.g., n is commonly 16 or 18, Molecular Cell Biology, supra. The term “unsaturated fatty acids” is used to denote, for example, the ¹H chemical shift of the CH₂ group at about 2.8 ppm, for example, as in —(CH═CH—CH₂—CH═CH)—, and/or the ¹H chemical shift of the unsaturated methinyl group —(CH═CH)— occurring at about 5.4 ppm. Alternatively, ¹³C resonances can be monitored for these groups. The ¹³C chemical shifts of the CH₂ group of saturated fatty acids are about 20.5, about 22.8, about 24.7, about 29.2-29.7, about 31.4, about 32 and about 34 ppm. ¹³C chemical shift of the inner double bonds —(CH═CH—CH₂—CH═CH)— group of unsaturated fatty acids is about 128 ppm. ¹³C chemical shift of the outer double bonds —(CH═CH)— and —(CH═CH—CH₂—CH═CH)— group of unsaturated fatty acids is about 130 ppm.

An embodiment of the present invention also provides for a method for monitoring interruption of protein translation in an animal having a cancerous tissue after administering a cancer therapy to the animal and measuring, by Magnetic Resonance Spectroscopy, the amount of saturated/unsaturated fatty acids present in the cancerous tissue before and after administering the cancer therapy. The current invention can be used for monitoring protein translation. Saturated/unsaturated fatty acids are metabolites of cellular membranes. The quantity of saturated/unsaturated fatty acids directly correlates with the amount of intracellular organelles, with intracellular membranes accounting for 90% of total cell membranes. Two molecular biology pathways add significance to the organelles, and therefore, significant impact to the Method. First, translation occurs on the surface of an organelle, the endoplasmic reticulum (ER), to generate secretory and integral proteins. The ribosomes are the workbenches of translation utilizing messenger RNA (mRNA) as a template to generate the correct amino acid (AA) sequence in the polypeptide. The nascent AA sequence leaves the ribosome and enters the lumen of the ER. Second, protein synthesis occurs within the lumen of organelles. Organelles are the machinery of protein synthesis. Protein synthesis of intergral and secretory proteins occurs within the lumens of the ER and Golgi apparatus (Ga). During the elongation of the polypeptides within the ER lumen, sulfur and carbohydrate side chains are added. The side chains provide bonds needed to maintain the fidelity of protein folding, within the ER and Ga. Two genetic pathways also add significance to the organelles, and therefore, significant impact to the Method. First, the abundance of organelles is controlled by the Unfolded Protein Response (UPR), a genetic pathway. The volume of organdies matches the level of protein synthesis by the Unfolded Protein Response (UPR). Bernales S, et al., Ann Rev Cell Dev Bio (2006) 22:487-508. The UPR is a signaling pathway between the organelles and DNA that control the ER volume, enzymes and chaperone proteins. Three receptors within the lumen of the ER monitor the fidelity of protein folding. When protein synthesis is amplified, and the ER cannot adequately fold the proteins, the receptors generate transcription factors that control 5% of the genes in the genome, thereby increasing ER volume, enzymen and chaperone proteins. The UPR also reduces the organelles when protein synthesis decreases, thereby maintaining the economy of metabolism. Second, protein synthesis is blocked in the early commitment phase of apoptosis, a genetic pathway. Apoptosis is genetically controlled programmed cell death. Schmitt C A, et al., J Pathol (1999) 187:127-137. All successful cancer therapies (cytostatic, antiangiogenesis and cytotoxic) cause programmed cell death. Early in the commitment phase of apoptosis translational initiation factors are blocked by phosphorylation and caspase degradation, thereby blocking assembly of ribosomes needed in translation/protein synthesis, Clemens M J, et al., Cell Death Differ (2000) 7:603-615. An increase in translation increases mass of these organelles and a decrease in translation decreases the mass of these organelles. Thus, the cellular saturated/unsaturated fatty acids level also reflects cellular protein synthesis. Accordingly, drugs down-regulating protein translation can be monitored by following an increase in the free saturated/unsaturated fatty acids levels. Therefore, the current invention can monitor early treatment response by quantifying an increase saturated/unsaturated fatty acids of degraded intracellular organelles linked to translation and protein synthesis. Further, the effect of drugs that can interrupt DNA replication or RNA transcription can be monitored by the current invention with interruption of protein translation visualized by increase in saturated/unsaturated fatty acids.

The amount of free saturated/unsaturated fatty acids can be measured by MRS in any suitable manner. For example, the amount of saturated/unsaturated fatty acids can be measured by measuring the height of a peak or peaks corresponding to saturated/unsaturated fatty acids. In another embodiment, the amount of saturated/unsaturated fatty acids can be measured by measuring the area under a peak or peaks corresponding to saturated/unsaturated fatty acids. In yet another embodiment, the amount of saturated/unsaturated fatty acids can be measured by measuring the ratio of the height of a peak or peaks corresponding to saturated/unsaturated fatty acids relative to the height of a peak or peaks of an internal or external standard. In a further embodiment, the amount of saturated/unsaturated fatty acids can be measured by measuring the ratio of the area under a peak or peaks corresponding to saturated/unsaturated fatty acids relative to the area under a peak or peaks of an internal or external standard.

Any suitable internal or external standard can be used. For example, the internal standard is total creatine when the MRS is based on ¹H resonance. The term “total creatine” refers to the combination of creatine and phosphocreatine. Creatine is buffered in cell systems; accordingly, the amount of creatine remains substantially constant. Examples of external standards are Electronic reference to access in vivo concentrations (ERETIC) and 3-(trimethylsilyl) propionic-2,2,3-d₄ acid (TSP). Alber M J, et al., Magn Reson Med 61, 525-532 (2009).

It is contemplated that the present inventive method is applicable to monitoring early treatment response wherein the treatment induces apoptosis.

In accordance with an embodiment of the present invention, any cancer therapy inducing apoptosis causes an interruption in an up-regulated intracellular organelle; for example, an interruption in the function of the secretory granules and/or the transporting vesicles. In accordance with another embodiment of the invention, any cancer therapy inducing apoptosis causes an interruption in the function of the Golgi apparatus. In further embodiments of the invention, inhibition of the cell surface receptor causes an interruption in the function of the lysosomes, the endoplasmic reticulum, the mitochondrion, the nucleus, and/or the peroxisomes.

The trafficking of the Choline polar heads and the saturated/unsaturated fatty acid tails of degraded endomembranes explains why choline decreases and saturated/unsaturated fatty acid tails increases during apoptosis. Choline, a small molecular essential nutrient, normally enters the cell by diffusion and by plasma membrane organic cation transporters. During apoptosis, choline decreases from both reduced active choline transport, and diffusion of choline out through the plasma membrane. Fatty acid tails are large chains, and are retained by intact plasma membrane as lipid droplets and within lysosomes; Kauppinen et al, supra.

A possible reason why the saturated/unsaturated fatty acid peaks increase before the Choline peak decreases is the continued active transport of choline into the cell until energy deprivation is complete.

In accordance with the present invention, any suitable cancer or tumor can be treated, for example, a cancer selected from the group consisting of brain cancer, colorectal cancer, breast cancer, acute leukemia, lung cancer, kidney cancer, squamous cell cancer, testicular cancer, stomach cancer, melanoma, sarcomas, ovarian cancer, non-small cell lung cancer, esophageal cancer, pancreatic cancer, neuroblastoma, mesothelioma, prostate cancer, bone cancer, kidney cancer, and hepatocellular cancer.

In accordance with the present inventive method, early treatment response can be measured within a period of about 168 hours, preferably about 24 hours, and more preferably about 12 hours, of the treatment. For example, the response can be monitored every 12, 24, 36, 48, 60, 72, 84, 96, 108, 120, 132, 144, 156, or 168 hours, or any combination thereof, after administration of the any cancer therapy.

Treatment response can also be documented using the current invention within 24 to 168 hours by monitoring protein translation. This embodiment utilizes a magnetic resonance magnet, equal to or greater than 0.5 Tesla, uses existing software and coils which are commercially available, has high spatial resolution, lacks radiation, employs user-friendly automatic sequences, allows non-invasive sequential analysis of drug doses/combinations, provides quantification from multi-sites, and can be employed with a plurality of drugs for trials. This embodiment can monitor treatment responses in vivo and in vitro, in humans and laboratory animals, as well as, in tissues and perfused cell extracts. This embodiment quantifies normal protein translation, as well as the amplified protein translation seen in cancer and inflammation. Since protein translation occurs in all cells, all cancer histologies can be studied. This embodiment is applicable to cytostatic (e.g., growth factor inhibitors), antiangiogenic therapies, and cytotoxic therapies (e.g., drugs and radiation). Potential uses of this embodiment include, but are not limited to, drug development and documenting interruption of signaling pathways.

The present invention also provides a method for monitoring cancer treatment comprising: (a) localizing a tumor in a patient; (b) selecting a region of interest (ROI) of the tumor; (c) obtaining magnetic resonance spectra (MRS) of the ROI; (d) measuring the amount of saturated/unsaturated fatty acids from the MRS spectra; (e) initiating treatment comprising administering a cancer therapy; (f) obtaining MR spectra of the tumor at the same ROI within a period of 7 days, preferably 3 days, and more preferably within 1 day, of initiating treatment; (g) measuring the amount of saturated/unsaturated fatty acids from the MR spectra; and (h) comparing the amount of saturated/unsaturated fatty acids obtained before treatment with the amount of saturated/unsaturated fatty acids obtained after treatment; whereby an increase in the amount of saturated/unsaturated fatty acids after treatment is indicative of a positive response to treatment.

The basis for clinical MR studies (e.g., MRS or MRI) is a nucleus, with a positive charge, for example, the hydrogen nucleus—the proton. The same machinery is used for MRI and MRS. They differ in the software manipulation of the emitted radiofrequency (RF) from the H nuclei. In MRI, the signal is used to create the image; in MRS, the signal is used to create the spectrum. Fourier Transform principle allows the MRS software to separate the individual RFs within the signal. The spectrum therefore represents the different RFs being emitted within the selected region of interest (ROI). The points along the horizontal axis of the spectrum represent specific RFs emitted from each metabolite. The vertical axis of the spectrum is proportional to the amount of each metabolite forming the area beneath the RF peaks. Spectra can be obtained on 0.5 T and higher MR scanners, although high-field strength scanners provide better definition of the spectra. Spectra obtained with different-strength scanners can be compared on a scale in parts per million (ppm) along the horizontal axis because metabolites always reside at one or more specific sites, for example, in ¹H MRS, the CH₂ of saturated fatty acids reside at 1.3 ppm, the CH₂ attached to methinyl carbons, e.g., as in —CH═CH—CH₂—CH=CH— groups, of unsaturated fatty acids reside at 2.8 ppm and the CH═CH ¹H resonance at 5.4 ppm.

Any suitable MR spectrometer can be used in the practice of the present invention. Clinical MR spectra can be obtained on MR scanners, for example, utilizing the clinical spectroscopy package called proton brain exam/single voxel (PROBE/SV) developed by General Electric Medical Systems (Milwaukee, Wis.) for use with GE's 1.5 Tesla (T) MR scanner. See Norfray, J. et al., supra, and Norfray, J. F. et al., supra, for procedures for obtaining MR spectra, identification of the peaks corresponding to metabolites such as Choline, creatine, and others, and ratio of the peaks. See also Danielsen and Ross, Magnetic Resonance Spectroscopy Diagnosis of Neurological Diseases, Marcel Dekker, Inc. (1999); Ross, B. et al., Magnetic Resonance Quarterly, 10, 191-247 (1994); and Ross et al., U.S. Pat. No. 5,617,861. Based on the information in the above publications, as well as information available in the art, those of skill in the art should be able to practice the invention on all types of tumors in accordance with the present invention.

The present invention can be carried out in any suitable manner, for example, as follows. Prior to initiating a therapy on a patient, the tumor is localized. Thus, for example, magnetic resonance images (MRIs) of the tumor, e.g., brain metastasis, breast malignancy, or bone tumor, with axial, sagittal, and coronal T1 and T2 images are obtained with and without contrast. A region of interest (ROI) of tumor is selected. This can be carried out based on the MRI findings to determine the tumor volume and location to be studied. MR spectra of the ROI are obtained within the tumor utilizing short and/or long TE (echo time) pulse sequences. The spectra obtained are interpreted. The peak corresponding to saturated fatty acids are identified, e.g., at a chemical shift of 1.3 ppm. To distinguish saturated fatty acids from a lactate peak at 1.33 ppm, lactate peak will have a doublet at short TE, and a lactate peak will invert below the baseline at a TE of 144 msec. Based on the saturated/unsaturated fatty acid peak(s), the amount of total cellular membranes is determined from either the height of the peak(s) or the area under the peak(s). An internal or external standard is identified in the ROI. An example of an internal standard is creatine or total creatine. An example of an external standard is 100% 2-(trimethylsilyl)ethanol (TSE), which may be taped to the head coil of the MR spectrometer. Kreis, R. et al., J. Magnetic Resonance, Series B 102, 9-19 (1993). The ratio of the saturated/unsaturated fatty acids to the standard is calculated. The saturated/unsaturated fatty acids to creatine ratio represent a measure of the total cell membranes within the ROI of the tumor prior to treatment.

The treatment of the tumor is initiated by administering an effective amount of the cancer therapy starting from time zero. The early treatment response can be monitored, for example, at 24 hours (day 1) to 168 hours (day 7), as follows. The tumor is localized utilizing the same MRI pulse sequences as prior to the treatment. The same ROI is selected within the tumor. MR spectra of the tumor are obtained utilizing the same pulse sequences, the same TR (relaxation time), TE (echo time), phases, and frequency averages. The MR spectra are interpreted as before and the saturated/unsaturated fatty acids to creatine ratios (e.g., height or area ratios) are calculated.

If the observed increase in the saturated/unsaturated fatty acids to creatine ratio is 15% or more, preferably 20% or more, and more preferably 25% or more relative to pre-treatment condition, then it can be concluded that an early response is positive and tumor regression has been achieved. The early increase in the saturated/unsaturated fatty acids to creatine ratio identifies a decrease in the intracellular cell membranes, for example, a decrease in the organelles and their granules and/or vesicles.

The present invention further provides a method for determining effectiveness of a molecule as a drug for treating cancer comprising administering an amount of the molecule to an animal having a cancerous tissue and measuring, by Magnetic Resonance Spectroscopy, the amount of saturated/unsaturated fatty acids present in the cancerous tissue before and after administering the molecule, wherein the molecule comprises a cancer therapy, whereby an increase in the amount of saturated/unsaturated fatty acids after administering the molecule is indicative of its effectiveness. The animals to be used in the present method can be, for example, mammals such as mice, rats, horses, guinea pigs, rabbits, dogs, cats, cows, pigs, and monkeys. The amount of cancer therapy will vary with a number of factors, e.g., weight of the animal, type of cancer, and severity of cancer, and is within the skill of the artisan. The potential drug can be administered by any suitable route of administration, e.g., oral, aerosol, parenteral, subcutaneous, intravenous, intraarterial, intramuscular, interperitoneal, rectal, and vaginal routes. The cancer can be natural or induced. Thus, effectiveness of a potential drug can be determined within a relatively short period of time, for example, within 12-168 hours, preferably 12-24 hours.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for monitoring early treatment response of a cancer treatment comprising measuring non-invasively, by magnetic resonance spectroscopy (MRS), the amount of saturated and/or unsaturated fatty acids present in the cancerous tissue before and after treatment, determining whether the amount of saturated and/or unsaturated fatty acids have increased, decreased, or remained the same, and correlating an increase in the amount of saturated and unsaturated fatty acids after treatment to a positive response resulting from interruption of protein translation due to the treatment.

2. The method of claim 1, wherein the MRS is based on the resonance of nuclei selected from the group consisting of ¹H, ¹³C, and any combination thereof.

3. The method of claim 2, wherein the MRS is based on ¹H resonance.

4. The method of claim 1, wherein measuring the amount of saturated/unsaturated fatty acid comprises measuring the height of a peak or peaks corresponding to saturated/unsaturated fatty acids.

5. The method of claim 1, wherein measuring the amount of saturated/unsaturated fatty acids comprises measuring the area under a peak or peaks corresponding to saturated/unsaturated fatty acids.

6. The method of claim 1, wherein measuring the amount of saturated/unsaturated fatty acids comprises measuring the ratio of the height of a peak or peaks corresponding to saturated/unsaturated fatty acids relative to the height of peak of an internal or external standard.

7. The method of claim 6, wherein the internal standard is total creatine or the methyl (CH₃) group of the fatty acids, wherein the MRS is based on ¹H resonance.

8. The method of claim 1, wherein measuring the amount of saturated/unsaturated fatty acids comprises measuring the ratio of the area under a peak or peaks corresponding to saturated/unsaturated fatty acids relative to the area under a peak of an internal or external standard.

9. The method of claim 8, wherein measuring the amount of saturated/unsaturated fatty acids comprises measuring the ratio of the area under a peak or peaks corresponding to saturated/unsaturated fatty acids relative to the area under a peak of an internal standard or an external standard.

10. The method of claim 9, wherein the internal standard is total creatine or the methyl (CH₃) group of the fatty acids, wherein the MRS is based on ¹H resonance.

11. The method of claim 1, wherein the amount of saturated or unsaturated fatty acids is measured by measuring the amount of a —CH₂— group resonance or a —CH═CH— group resonance.

12. The method of claim 11, wherein the ¹H resonance of the —CH₂— group of a saturated fatty acid is measured at about 1.3 ppm and/or the —CH₂— group next to an unsaturated —CH═CH— group is measured at about 2.8 ppm.

13. The method of claim 11, wherein the ¹³C resonance of the —CH₂— group is measured at a position selected from the group consisting of about 20.5, about 22.8, about 24.7, about 29.2-29.7, about 31.4, about 32 and about 34 ppm.

14. The method of claim 11, wherein the ¹H resonance of the —CH═CH— group is measured at about 5.4 ppm.

15. The method of claim 11, wherein the ¹³C resonance of the —CH═CH— group is measured at about 128 ppm or about 130 ppm.

16-29. (canceled)

30. A method for monitoring cancer treatment comprising: (a) localizing a tumor in a patient; (b) selecting a region of interest (ROI) of the tumor; (c) obtaining magnetic resonance spectra (MRS) of the ROI; (d) measuring the amount of saturated/unsaturated fatty acids from the MRS spectra; (e) initiating treatment comprising administering a cancer therapy; (f) obtaining MR spectra of the tumor at the same ROI within a period of 7 days of initiating treatment; (g) measuring the amount of saturated/unsaturated fatty acids from the MR spectra; and (h) comparing the amount of saturated/unsaturated fatty acids obtained before treatment with the amount of saturated/unsaturated fatty acids obtained after treatment; and correlating an increase in the amount of saturated/unsaturated fatty acids after treatment with a positive response to treatment.

31-32. (canceled)

33. A method for determining effectiveness of a molecule as a drug for treating cancer comprising administering an amount of the molecule to an animal having a cancerous tissue and measuring non-invasively, by Magnetic Resonance Spectroscopy, the amount of saturated/unsaturated fatty acids present in the cancerous tissue before and within a period of about 168 hours after administering the molecule, obtaining a difference between the saturated/unsaturated fatty acids amounts, and correlating the increase in the saturated/unsaturated fatty acids amounts with effectiveness of the cancer treatment or lack thereof.

34. A method for monitoring interruption of protein translation in an animal having a cancerous tissue comprising administering a cancer therapy to the animal and measuring, by Magnetic Resonance Spectroscopy, the amount of saturated/unsaturated fatty acids present in the intracellular membranes of the cancerous tissue before and after administering the cancer therapy, obtaining a difference between the amounts of saturated/unsaturated fatty acids, and correlating the increase to interruption of protein translation.

35. A method for determining the effectiveness of a cancer treatment to induce apoptosis from interruption of protein translation in an animal having a cancerous tissue comprising administering an amount of the molecule to an animal having a cancerous tissue and measuring non-invasively, by Magnetic Resonance Spectroscopy, the amount of saturated/unsaturated fatty acids present in the cancerous tissue before and within a period of about 168 hours after administering the molecule, obtaining a difference between the saturated/unsaturated fatty acids amounts, and correlating the increase in the saturated/unsaturated fatty acids amounts with effectiveness of the cancer treatment or lack thereof.

36. The method of claim 1, wherein measuring the amount of saturated/unsaturated fatty acids comprises measuring the amount of saturated (CH₂) resonance and/or amount of the unsaturated (CH═CH) resonance.
